(19) United States
(12) Patent Application Publication
(10) Pub. No.: US 2022/0229026 A1
(43) Pub. Date: Jul. 21, 2022

(54) METHODS OF DETECTING GLYCOGEN AND POLYGLUCAN

(71) Applicant: University of Kentucky Research Foundation, Lexington, KY (US)

(72) Inventors: Matthew S. Gentry, Lexington, KY (US); Ronald Bruntz, Lexington, KY (US); Ramon C. Sun, Lexington, KY (US)

(21) Appl. No.: 17/580,513

(22) Filed: Jan. 20, 2022

Related U.S. Application Data
(60) Provisional application No. 63/139,615, filed on Jan. 20, 2021.

(51) Int. Cl. G01N 30/72; G01N 33/66; H01J 49/16; H01J 49/00; H01J 49/26

(52) U.S. Cl. CPC G01N 30/7206; G01N 33/66; H01J 49/164; H01J 49/0031; G01N 2030/025; G01N 2333/944; G01N 2400/48; G01N 2570/00; H01J 49/26

(57) ABSTRACT

Provided herein are methods of measuring glycogen and methods of diagnosing a disease. One method of measuring includes separating sugar monomers and sugar phosphates using gas-chromatography, and analyzing the monomers and phosphates using mass spectrometry. Another method of measuring includes adding an isoamylase to a sample, the isoamylase cleaving glucose chains from glycogen; applying a matrix-assisted laser desorption ionization (MALDI) ionization matrix to the sample; and analyzing the samples using matrix-assisted laser desorption ionization mass spectrometry (MALDI-MS). The method of diagnosing a disease includes determining an amount and location of glycogen accumulation in a subject; and diagnosing a disease when over-accumulation of glycogen is determined.

### RELATED APPLICATIONS

This application claims priority from U.S. Provisional Application Ser. No. 63/139,615, filed Jan. 20, 2021, the entire disclosure of which is incorporated herein by this reference.

### GOVERNMENT INTEREST

This invention was made with government support under grant numbers R01AG06665, NS070899, NS116824 and NS070899-0952 awarded by the National Institutes of Health (NIH). The government has certain rights in the invention.

### TECHNICAL FIELD

The present disclosure is directed to articles and methods for measuring glycogen. In particular, the disclosure is directed to articles and methods for measuring normal or diseased glycogen in mammalian tissues and biofluids as a diagnostic tool and a biomarker to track disease progression for various diseases.

### BACKGROUND

Glycogen is a class of naturally occurring sugar polymers in the human body that has been reported in virtually all tissues and subject to microenvironmental influence. The current gold-standard to quantitate and visualize glycogen in situ is Periodic acid-Schiff (PAS), a method invented in 1948. Due to limited sensitivity and cross reactivity to other polysaccharides, PAS has limited applications only in glycogen storage diseases and a few unique classes of cancers. Without a more sensitive and robust assay with spatial resolution it is challenging to study the functional roles of glycogen in situ. Accordingly, there remains a need in the art for methods of detecting glycogen with increased sensitivity and specificity.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

In some embodiments, the presently-disclosed subject matter is directed to a method for measuring glycogen, the method including separating sugar monomers and sugar phosphates using gas-chromatography, and analyzing the monomers and phosphates using mass spectrometry. In some embodiments, the gas-chromatography is coupled to the mass spectrometry. In some embodiments, the method provides measurement of sugar monomers and sugar phosphates with femtogram detection limit in any suitable fluid or sample.

Also provided herein, in some embodiments, is a method for measuring glycogen in healthy and diseased tissue, the method including adding an isoamylase to a sample, the isoamylase cleaving glucose chains from glycogen; applying a matrix-assisted laser desorption ionization (MALDI) ionization matrix to the sample; and analyzing the samples using matrix-assisted laser desorption ionization mass spectrometry (MALDI-MS). In some embodiments, the isoamylase cleaves glucose chains at only the α1,-6 linkages. In some embodiments, the method further includes releasing N-linked glycans. In some embodiments, releasing N-linked glycans includes adding peptide-N-glycosidase F (PNGase F) to the sample. In some embodiments, the MALDI ionization matrix is selected from the group consisting of α-cyano-4-hydroxycinnamic acid (CHCA) and DHB. In some embodiments, analyzing the sample using MALDI-MS includes analyzing the sample with or without an ion-mobility enabled mass spectrometer.

Further provided herein, in some embodiments, is a method of diagnosing a disease, the method including determining an amount and location of glycogen accumulation in a subject; and diagnosing a disease when over-accumulation of glycogen is determined. In some embodiments, the determining step includes measuring the amount glycogen.

In some embodiments, the measuring includes separating sugar monomers and sugar phosphates using gas-chromatography; and analyzing the monomers and phosphates using mass spectrometry. In some embodiments, the gas-chromatography is coupled to the mass spectrometry. In some embodiments, the method provides measurement of sugar monomers and sugar phosphates with femtogram detection limit in any suitable fluid or sample.

In some embodiments, the measuring includes adding an isoamylase to a sample, the isoamylase cleaving glucose chains from glycogen; applying a matrix-assisted laser desorption ionization (MALDI) ionization matrix to the sample; and analyzing the samples using matrix-assisted laser desorption ionization mass spectrometry (MALDI-MS). In some embodiments, the isoamylase cleaves glucose chains at only the α1,-6 linkages. In some embodiments, the method further includes releasing N-linked glycans. In some embodiments, releasing N-linked glycans includes adding peptide-N-glycosidase F (PNGase F) to the sample. In some embodiments, the MALDI ionization matrix is selected from the group consisting of α-cyano-4-hydroxycinnamic acid (CHCA) and DHB. In some embodiments, analyzing the sample using MALDI-MS includes analyzing the sample with or without an ion-mobility enabled mass spectrometer.

Further features and advantages of the presently-disclosed subject matter will become evident to those of ordinary skill in the art after a study of the description, figures, and non-limiting examples in this document.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIGS. 1A-E show images and graphs illustrating MALDI-mass spectrometry imaging of glycogen and N-glycans. (A) Schematic of the workflow for dual imaging of glycogen and N-glycans. Slides are treated with peptide-N-glycosidase F (PNGase F) to release N-linked glycans, or isoamylase to cleave α-1,6-glycosidic bonds releasing linear oligosaccharide chains, or both. After adding the α-cyano-4-hydroxycinnamic acid (CHCA) ionization matrix for MALDI, samples were analyzed by MALDI and traveling wave ion mobility mass spectrometry (TW IMS). (B) Representative ion chromatogram of linear polysaccharide chains derived from glycogen based on ion mobility separation. Only chain lengths of 9-15 are shown. (C) Representative ion chromatogram of released N-glycans based on ion mobility separation, structures of the most abundant glycans are shown as diagrams. (D) MALDI-MSI displaying regional and relative abundance of glycogen and N-glycans in WT and LKO mouse brain sagittal sections. Glycogen was quantified by combining linear chain polysaccharides between 4-15 sugar monomers long. N-glycans were quantified by combining the intensity of the three most abundant N-glycans with molecular masses of 1257, 1419, and 1688. Images display a gradient of the abundance for each. (E) Images display the same data as D but presented as red for glycogen and blue for N-glycans. (F) Immunohistostaining with an established anti-PGB antibody in wild-type (WT) and LKO mouse brain.

FIGS. 2A-C show a graph and images illustrating ion mobility separation of glycogen and N-glycans by MALDI-TW MSI. (A) Scatter plot of monoisotopic mass versus drift time in the ion mobility cell for N-glycans, linear polysaccharides (chain length), and the MALDI matrix. (B) Spatial distribution of glucose chains of 9 and 15 monomers long in WT and LKO mice. (C) Spatial distribution of N-glycans separating at 1257 m/z and 1485 m/z in WT and LKO mice. Glycan structures are placed on the right side of the corresponding image. Intensity and size scales are located beneath the images.

FIGS. 3A-I Additional example of MALDI-TW MSI of glycogen in a mouse liver. (A) Schematic of the workflow for imaging of glycogen. Slides are treated with isoamylase to cleave α-1,6-glycosidic bonds releasing linear oligosaccharide chains. After adding the α-cyano-4-hydroxycinnamic acid (CHCA) ionization matrix for MALDI, samples were analyzed by MALDI and traveling wave ion mobility mass spectrometry (TW IMS). (B) Representative ion chromatogram of linear polysaccharide chains derived from glycogen based on ion mobility separation. Only chain lengths of 3-13 are shown. (C) MALDI images of mouse liver with different glucose polymers. (D) Schematic of the workflow for co-imaging of glycogen and glycogen in the same slice of mouse liver in (A). (E) 2D ion mobility separation of glycogen and glycan based on mass differences and drift time. (F) Histological image of mouse liver. (G) overlay of glycan and glycogen of the same mouse liver produced from MALDI-MSI. (H) glucose polymers isolated from ion mobility separation (I) Glycans separated from ion mobility separation.

FIGS. 4A-G show MALDI imaging and detection of glycogen in human liver slices. (A) histological image of human liver slice. (B) glucose polymer distribution of glycogen detected in liver. (C) glycogen distribution in the liver slice shown in (A). (D) phosphorylated glucose polymers of liver glycogen detected by MALDI-MSI. (E) glycogen and glycan overlay in liver tissue from (A) (F) ratio of phospho-glucose polymers and unphosphorylated glucose polymers detected by MALDI-MSI. (G) glycogen structure based on data presented in (A)-(F).

FIGS. 5A-Q show MALDI imaging and detection of glycogen in prostate, lung cancer, and Ewing sarcoma patient tumors. (A)-(D) histological images of prostate, lung squamous cell carcinoma (LSCC), lung adenocarcinoma (LUAD), and Ewing's sarcoma. (E)-(H) MALDI images of glycogen and glycan overlap for each of the tissue slices shown in (A)-(D). (I)-(L) Glycogen only distribution in tissue slices shown in A-D using MALDI-MSI. (M)-(P) Glycogen derived glucose polymers detected from tissue slices shown in (A)-(D) detected by MALDI-MSI. (Q) graphical representation of glycogen size and abundance between tissue types shown in (A-D).

FIGS. 6A-K show MALDI imaging and detection of glycogen in normal and AD brains. (A) and (D) histological image of aged human and AD human patients. (B) and (E) MALDI MSI of glycogen in aged and AD human brain specimens. (C) and (F) Overlay of glycogen and glycan in both aged and AD human brain specimens. (G) Glycogen derived glucose polymers in the grey matter of aged and AD human specimens. (H) Glycogen derived glucose polymers in the white matter of aged and AD human specimens. (I) Glycogen derived phospho-glucose polymers in the grey matter of aged and AD human specimens. (J) Glycogen derived phospho-glucose polymers in the white matter of aged and AD human specimens. (K) graphical representation of glycogen size and abundance between normal ages and AD brains.

FIGS. 7A-O show MALDI detection of glycogen content and regional distribution in a human Ewing's sarcoma specimens. (A) Histological images of Ewing's sarcoma resected from shoulder tissue from surgery. (B) Glycogen only MALDI images of Ewing's sarcoma resected from shoulder tissue from surgery. (C) Glycogen and glycan overlay of MALDI images from Ewing's sarcoma resected from shoulder tissue from surgery. (D) Histological images of Ewing's sarcoma resected from chest wall tissue from surgery. (E) Glycogen only MALDI images of Ewing's sarcoma resected from chest wall tissue from surgery. (F) Glycogen and glycan overlay of MALDI images from Ewing's sarcoma resected from chest wall tissue from surgery. (G) Histological images of Ewing's sarcoma resected from rib tissue from surgery. (H) Glycogen only MALDI images of Ewing's sarcoma resected from rib tissue from surgery. (I) Glycogen and glycan overlay of MALDI images from Ewing's sarcoma resected from rib tissue from surgery. (J) Histological images of Ewing's sarcoma resected from abdomen tissue from surgery. (K) Glycogen only MALDI images of Ewing's sarcoma resected from abdomen tissue from surgery. (L) Glycogen and glycan overlay of MALDI images from Ewing's sarcoma resected from abdomen tissue from surgery. (M) Histological images of Ewing's sarcoma resected from testis tissue from surgery. (N) Glycogen only MALDI images of Ewing's sarcoma resected from testis tissue from surgery. (O) Glycogen and glycan overlay of MALDI images from Ewing's sarcoma resected from testis tissue from surgery.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DEFINITIONS

Unless defined otherwise, all technical and scientific terms used herein have the same meaning as commonly understood by one of ordinary skill in the art to which the disclosure belongs. Any methods and materials similar to or equivalent to those described herein can be used in the practice or testing of the present disclosure, including the methods and materials are described below.

Following long-standing patent law convention, the terms “a,” “an,” and “the” refer to “one or more” when used in this application, including the claims. Thus, for example, reference to “a liquid” includes a plurality of liquids, and so forth.

The terms “comprising,” “including,” and “having” are intended to be inclusive and mean that there may be additional elements other than the listed elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration, percentage, or the like is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E1Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used herein, the terms “optional” or “optionally” means that the subsequently described event or circumstance can or cannot occur, and that the description includes instances where said event or circumstance occurs and instances where it does not.

Unless explicitly stated otherwise, as used herein, the term “glycogen” refers to both normal glycogen and diseased glycogen, which is also called polyglucans or polyglucosan bodies.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

Provided herein are methods for detecting and/or measuring glycogen. For example, in some embodiments, the methods include detecting glycogen storage in organs such as, but not limited to, liver, lung, bladder, testis, heart, kidney, and the brain. In some embodiments, the method includes measuring sugar monomers and sugar phosphates using gas-chromatography mass spectrometry. For example, in some embodiments, the method includes first separating sugar monomers and sugar phosphates using gas-chromatography, and then analyzing the monomers and phosphates using mass spectrometry. Suitable sugar monomers include, but are not limited to, glucose, mannose, fucose, galactose, or glucosamine. Suitable sugar phosphates include, but are not limited to, glucose phosphate, mannose phosphate, fucose phosphate, galactose phosphate, glucosamine phosphate.

The gas-chromatography may be performed with any suitable gas-chromatograph. Similarly, the mass spectrometry may be performed with any suitable mass spectrometer. Suitable mass spectrometers include, but are not limited to, single quadrupole, triple quadrupole, time-of-flight, or Orbitrap. In some embodiments, the gas-chromatograph is coupled to the mass spectrometer. The gas-chromatography mass spectrometry methods disclosed herein provide measurement of sugar monomers and sugar phosphates with femtogram detection limit in any suitable fluid or sample. Suitable fluids or samples include, but are not limited to, biofluids such as urine, cerebrospinal fluid (CSF), plasma, or any other suitable biofluid where measurement of glycogen is desired.

In some embodiments, the method includes measuring glycogen through matrix-assisted laser desorption ionization mass spectrometry (MALDI-MS), such as matrix-assisted laser desorption/ionization-mass spectrometry imaging (MALDI-MSI), a new technique in analytical chemistry that can be used to profile biological features with spatial distribution information. In some embodiments, the method includes a workflow utilizing MALDI for the detection and visualization of glycogen in healthy and diseased tissues. In some embodiments, the method includes first cleaving glucose chains from glycogen by the addition of isoamylase, then applying the MALDI ionization matrix, followed by analyzing the samples using MALDI.

The glucose chains may be cleaved from the glycogen using any suitable isoamylase. In some embodiments, the isoamylase includes any suitable enzyme isoamylase that specifically cleaves glucose chains at only the α1,-6 linkages. In some embodiments, the method includes dual imaging of glycogen and N-glycans. In such embodiments, the method also includes releasing N-linked glycans with any suitable enzyme, such as, but not limited to, peptide-N-glycosidase F (PNGase F). The MALDI ionization matrix includes any suitable ionization matrix that is compatible with MALDI. Suitable ionization matrixes include, but are not limited to, α-cyano-4-hydroxycinnamic acid (CHCA) or DHB for detection of N-linked glycans by MALDI-MSI. The enzyme(s) and MALDI matrix may be applied simultaneously or sequentially using any suitable method of application, such as, but not limited to, uniform application using a high velocity dry-spraying robot.

Once the enzymes and MALDI matrix have been applied, the released linear glucose chains are analyzed using a standard or ion-mobility enabled mass spectrometer (e.g., Orbitrap, FTMS, QTOF). In some embodiments, ion-mobility provides improved glucose chain detection as compared to other mass spectrometers by separating glucose chains from ionization matrix based on differential collision cross section. In some embodiments, ionization of matrix and glycogen is performed using a high-power high-energy UV laser. In one embodiment, directing a pulsed laser beam towards the sample causes desorption of the sample and matrix material, and breaks down the matrix material to produce gas phase ionic species. The analyte molecules are ionized via protonation or de-protonation, and then accelerated into a mass spectrometer system for mass analysis.

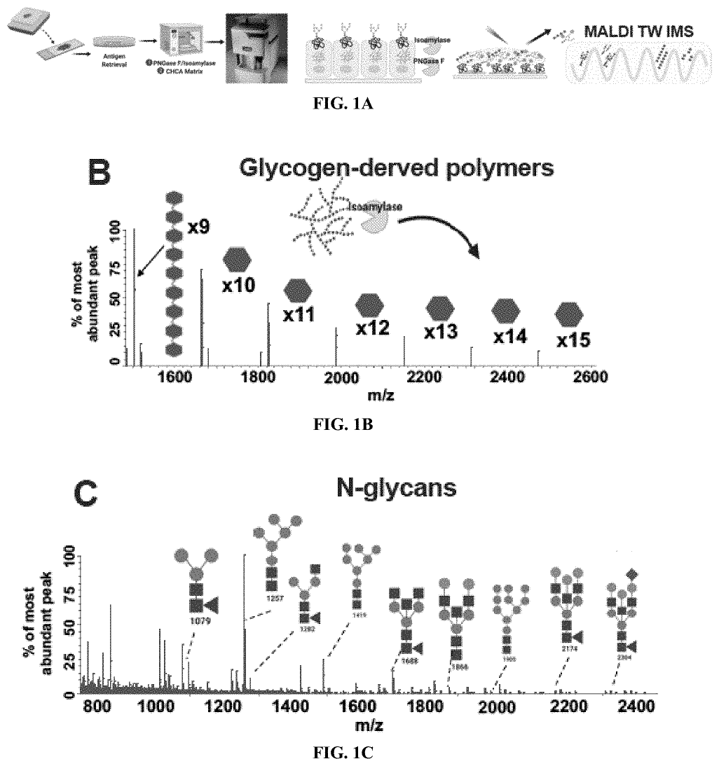

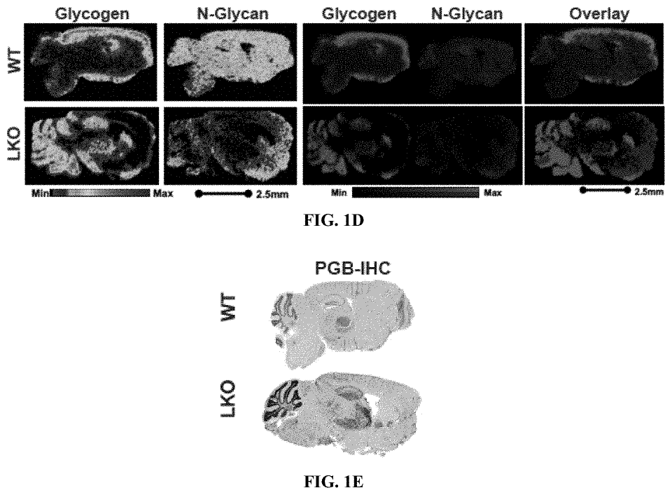

Referring to FIG. 1A, in one example, dual imaging of glycogen and N-glycans includes first treating slides with peptide-N-glycosidase F (PNGase F) to release N-linked glycans, or isoamylase to cleave α-1,6-glycosidic bonds releasing linear oligosaccharide chains, or both. Next, α-cyano-4-hydroxycinnamic acid (CHCA) ionization matrix for MALDI is added and then the samples are analyzed by MALDI and traveling wave ion mobility mass spectrometry (TW IMS). As will be appreciated by those skilled in the art, the example above is provided for illustration purposes only and the methods disclosed herein are not limited thereto. For example, the MALDI-MS methods disclosed herein may be either standalone or enzyme-assisted. In some embodiments, the MALDI-MS methods disclosed herein are capable of assessing sugar polymers from 2-50 units long with spatial resolution of under 50 μm in tissues, tissue microarray, cell lines grown in chamber wells, biofluids, or any other suitable sample.

Also provided herein are methods of detecting and/or diagnosing various diseases. In some embodiments, the method includes measuring glycogen according to one or more of the embodiments disclosed herein, determining an amount and location of glycogen accumulation, and detecting/diagnosing a disease when over accumulation of glycogens is determined. Additionally or alternatively, the method may include assessing progression of the disease and/or response of the disease to treatment by comparing the determined amount of glycogen accumulation. In some embodiments, the disease includes, but is not limited to, Alzheimer's disease related dementias (ADRD), spinal cord injury, Traumatic brain injury, lafora disease, glycogen storage diseases type I-IV, adult polyglucosan diseases, ALS, lung cancer, bladder cancer, colon cancer, and pancreatic cancer, or glycogen-rich class of tumors (e.g., breast, bone, bladder, renal, and liver). Other diseases include, but are not limited to, any disease where accumulation of glycogen serves as a biomarker for detection and/or progression. Unlike existing methods, such as Periodic Acid-Schiff (PAS) staining, which has very limited sensitivity and specificity, the method disclosed herein provide significantly more sensitive glycogen measurement. For example, in some embodiments, the methods disclosed herein provide 50-fold to 100,000-fold more sensitive detection of glycogens, as compared to existing methods.

The presently-disclosed subject matter is further illustrated by the following specific but non-limiting examples. The following examples may include compilations of data that are representative of data gathered at various times during the course of development and experimentation related to the presently-disclosed subject matter. Those skilled in the art will recognize, or be able to ascertain, using no more than routine experimentation, numerous equivalents to the specific substances and procedures described herein.

### EXAMPLES

### Example 1—Brain Glycogen Serves as a Critical Glucosamine Cache Required for Protein Glycosylation

Glycosylation defects are a hallmark of many nervous system diseases. However, the molecular and metabolic basis for this pathology are not fully understood. This Example discusses the finding that N-linked protein glycosylation in the brain is coupled to glucosamine metabolism through glycogenolysis. It was discovered that glucosamine is an abundant constituent of brain glycogen, which functions as a glucosamine reservoir for glycosylation precursors. The incorporation of glucosamine into glycogen by glycogen synthase and release by glycogen phosphorylase in vitro was defined by biochemical and structural methodologies, in situ in primary astrocytes, and in vivo by isotopic tracing and mass spectrometry. Using mouse models of two glycogen storage diseases, it was shown that disruption of brain glycogen metabolism causes global decreases in free UDP-N-acetyl-glucosamine and N-linked protein glycosylation. These findings revealed key fundamental biological role for brain glycogen in protein glycosylation with direct relevance to multiple human diseases of the central nervous system.

Protein Glycosylation Defects in Brain Regions with PGBs in LKO Mice

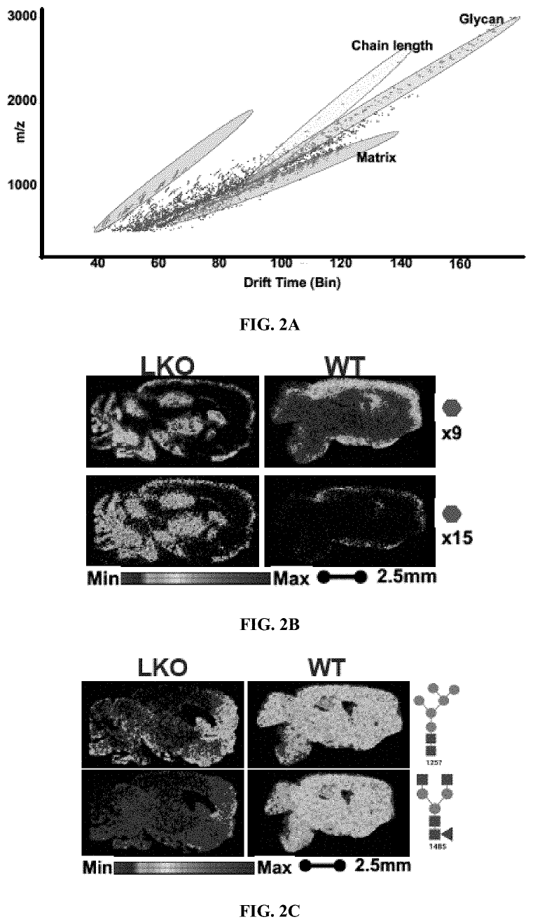

GlcNAc is the basic building block for the initiation of N-glycan biosynthesis.¹ The present inventors' results with the LKO mouse brains indicated that by sequestering glycogen, PGBs impair protein N-glycosylation, which would be evident by a reduction in N-glycan abundance. A novel workflow was developed to evaluate the relationship between PGBs, glycogen, and N-glycan abundance in specific brain regions. Matrix-assisted laser desorption/ionization (MALDI) coupled with traveling-wave ion-mobility high-resolution mass spectrometry (TW IMS) was used (FIG. 1A). Formalin-fixed paraffin-embedded brain sections were treated with peptide-N-glycosidase F (PNGase F) and isoamylase applied by a high-velocity robotic sprayer. PNGase F releases protein-bound N-linked glycans, and isoamylase cleaves the glycogen α-1,6-glycosidic bonds to release linear oligosaccharide chains from 3-25 glucose units in length. TW IMS separates oligosaccharides and N-linked glycans based on differential collision cross section² (FIG. 2A). With this method, glycogen was quantitatively imaged by combining linear chain polysaccharides between 4-15 sugar monomers, and specific N-glycans of various compositions within the brain sections (FIGS. 1B-C).

Using this method, the distribution of glycogen and N-glycans was compared in brains of wild-type and LKO mice (FIGS. 1D-E and 2B-C). As a validation of the method, immunohistochemistry was also performed with antibodies recognizing PGBs (FIG. 1E). In wild-type mice, glycogen was most abundant in the frontal cortex (FIGS. 1D-E). In contrast, the LKO mice had abundant glycogen in the cerebellum, hindbrain, midbrain, and the hippocampus (FIGS. 1D-E). This PGB distribution was similar to previous reported.³⁻⁴ In addition to increased glycogen, the LD mouse brains exhibited decreases in three of the most abundant N-linked mouse brain glycans (FIGS. 1C-D and 2C). An overlay of glycogen and glycan MALDI images revealed dramatic reductions of N-linked glycans in areas of PGB (glycogen) accumulation (FIG. 1E). The glycan profiling was expanded using a fourier-transform mass spectrometer (FTMS) with sufficient sensitivity to detect larger N-glycan structures⁵. Throughout the LD mouse brain, decreased N-linked glycans of most structures was observed (FIGS. 2A-C).

To assess if altered glycosylation correlated with cellular responses, immunohistochemical analyses were performed to evaluate XPB1 and GRP78, markers of the unfolded protein response (UPR) and ER stress. The analyses focused on the hippocampus, brainstem, and thalamus, because these were regions with high PGB levels (FIG. 1D).

### Example 2—Visualizing Glycogen Metabolism In Situ by Mass Spectrometry Imaging

Glycogen is a direct modulator of cellular metabolism, molecular processes, and organismal physiology. Dysregulation in glycogen metabolism is observed during aging, and ectopic glycogen accumulation drives metabolic dysregulation, ER stress, and apoptosis in tumorigenesis and neurodegeneration. Glycogen consist of branched carbohydrates arranged into concentric patterns though the strategic placement of phosphates. Due to its unique physiochemical properties, glycogen is extremely hard to measure biochemically and visualize in situ. The current gold-standard to quantitate and visualize glycogen in situ is PAS. However, due to low sensitivity, PAS has limited applications only in glycogen storages and few unique classes of cancers. As such, while glycogen has been reported in virtually all tissues and subject to microenvironmental influence, it is challenging to study the functional roles of glycogen in situ without a more sensitive and robust assay with spatial resolution.

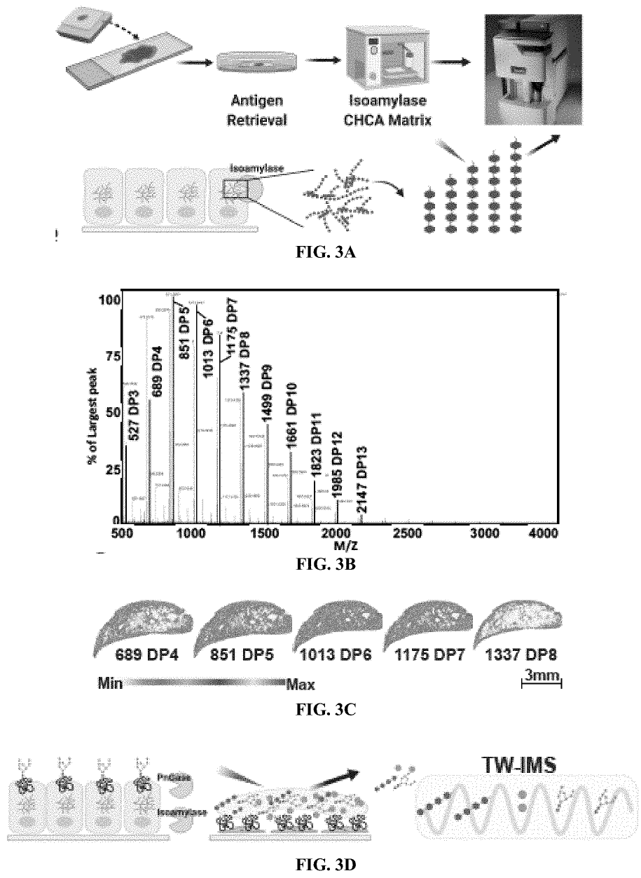

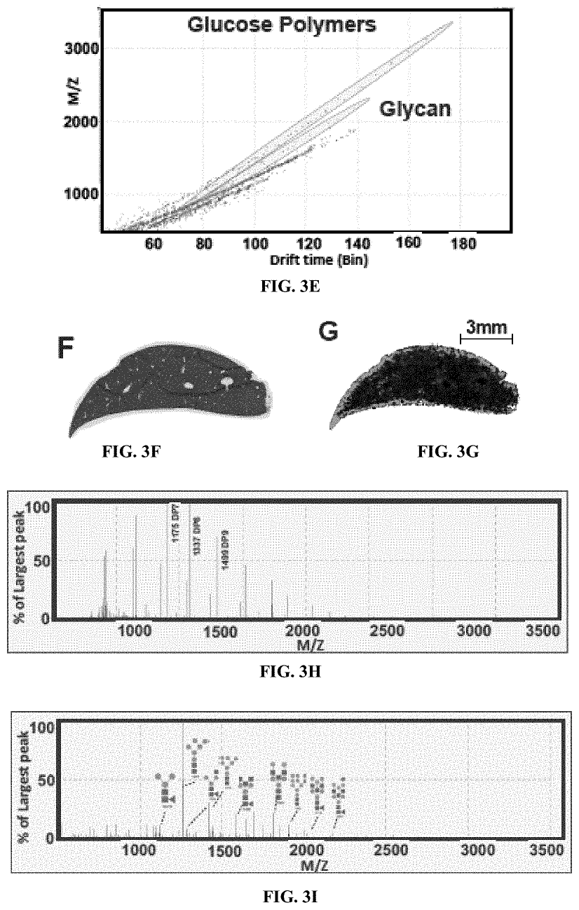

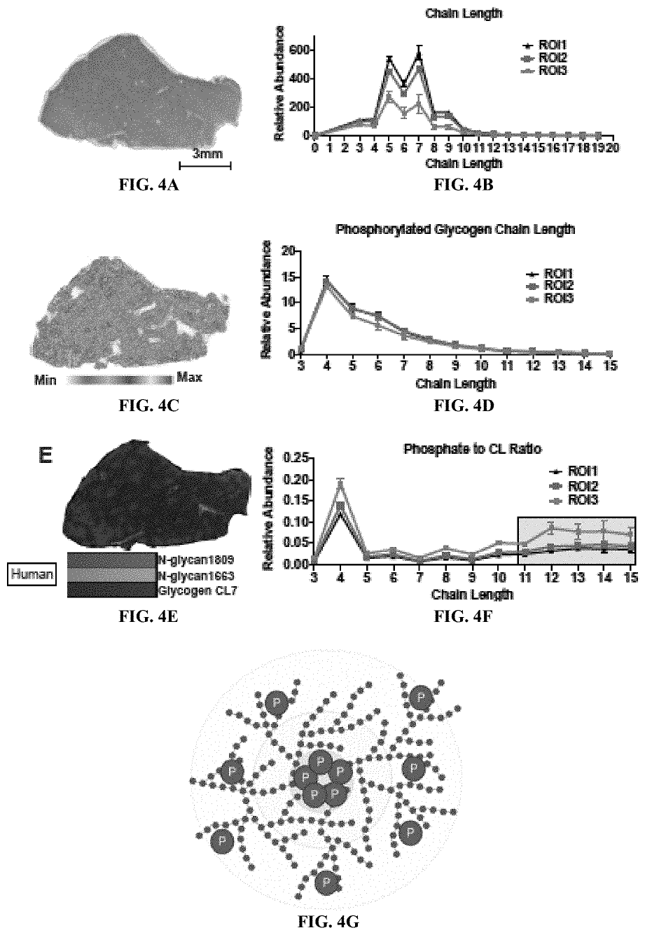

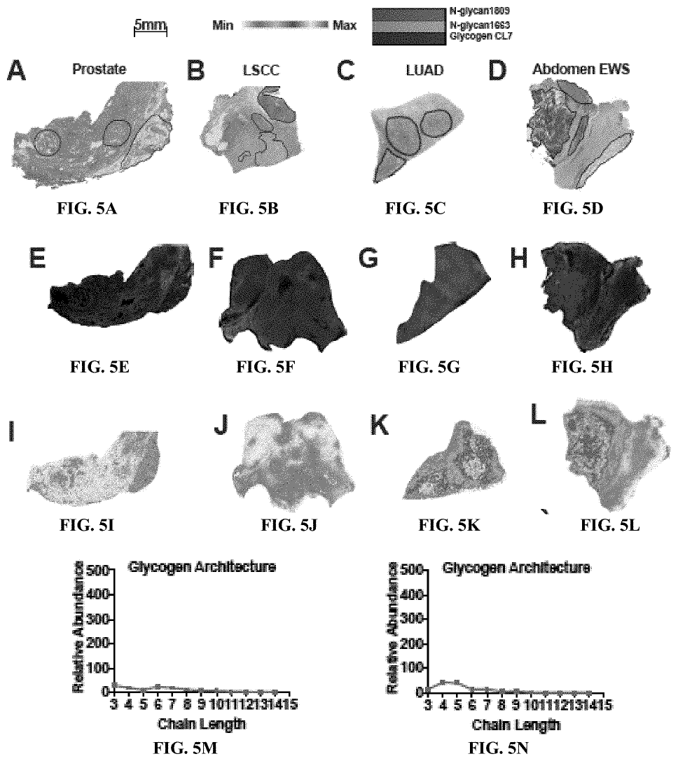

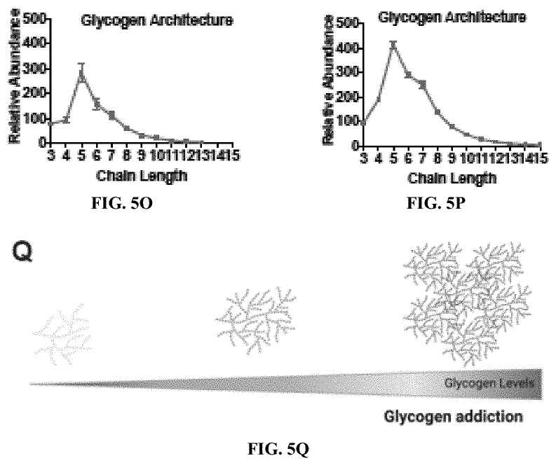

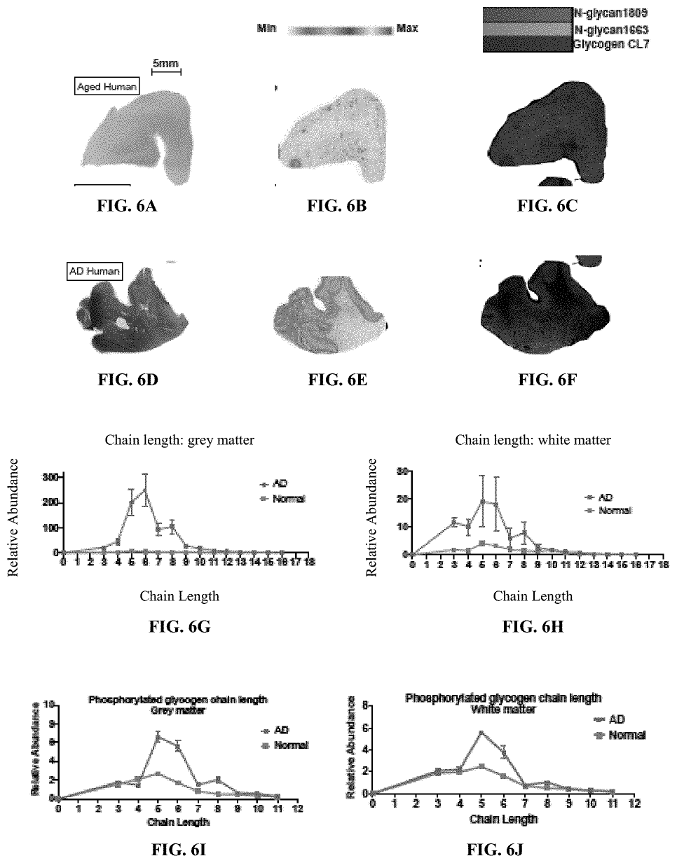

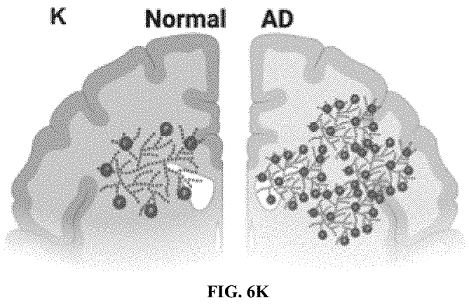

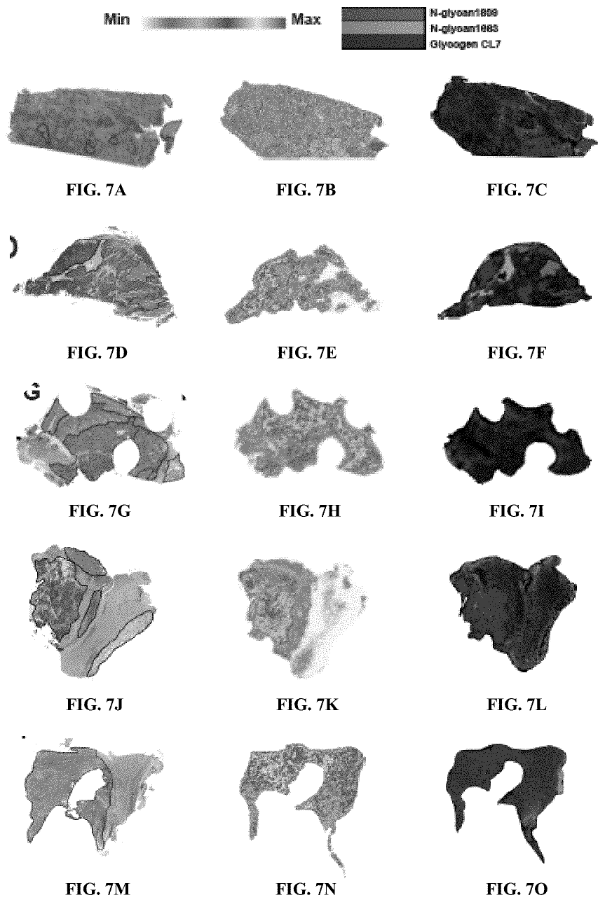

Referring to FIGS. 3A-7O, this Example describes a new method to image microenvironmental glycogen utilizing enzyme assisted release of glycogen substrates couple to MALDI mass spectrometry imaging. The assay disclosed herein provides robust information on localization and heterogeneity of glycogen in brain, liver, kidney, testis, lung, bladder, and bone in both mouse and human tissues. This assay was applied to study glycogen in terminal disease including frontal cortex of normal, AD specimens, and different cancer subtypes. The results demonstrate that glycogen accumulation is regional and diseases specific. Additionally, it was discovered that a gradient of glycogen accumulation exists among different tumors of different origin. Furthermore, evidence is provided that the growth of Ewing's sarcoma, a glycogen-dependent, pediatric bone cancer, is completely ablated in vivo through pharmacological and genetic intervention against glycogen accumulation.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Stanley et al., 2017.
2. Huang and Dodds, 2013.
3. Ganesh et al., 2002.
4. Yokota et al., 1988.
5. Powers et al., 2013.
6. López-González et al., 2017.
7. Deslauriers et al., 2011.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

What is claimed is:

1. A method for measuring glycogen, the method comprising:
separating sugar monomers and sugar phosphates using gas-chromatography; and
analyzing the monomers and phosphates using mass spectrometry.

2. The method of claim 1, wherein the gas-chromatography is coupled to the mass spectrometry.

3. The method of claim 1, wherein the method provides measurement of sugar monomers and sugar phosphates with femtogram detection limit in any suitable fluid or sample.

4. A method for measuring glycogen in healthy and diseased tissue, the method comprising:
adding an isoamylase to a sample, the isoamylase cleaving glucose chains from glycogen;
applying a matrix-assisted laser desorption ionization (MALDI) ionization matrix to the sample; and
analyzing the samples using matrix-assisted laser desorption ionization mass spectrometry (MALDI-MS).

5. The method of claim 4, wherein the isoamylase cleaves glucose chains at only the α1,-6 linkages.

6. The method of claim 4, further comprising releasing N-linked glycans.

7. The method of claim 6, wherein releasing N-linked glycans comprises adding peptide-N-glycosidase F (PNGase F) to the sample.

8. The method of claim 4, wherein the MALDI ionization matrix is selected from the group consisting of α-cyano-4-hydroxycinnamic acid (CHCA) and DHB.

9. The method of claim 4, wherein analyzing the sample using MALDI-MS includes analyzing the sample with or without an ion-mobility enabled mass spectrometer.

10. A method of diagnosing a disease, the method comprising:
determining an amount and location of glycogen accumulation in a subject; and
diagnosing a disease when over-accumulation of glycogen is determined.

11. The method of claim 10, wherein the determining step includes measuring the amount glycogen.

12. The method of claim 11, wherein the measuring comprises separating sugar monomers and sugar phosphates using gas-chromatography; and
analyzing the monomers and phosphates using mass spectrometry.

13. The method of claim 12, wherein the gas-chromatography is coupled to the mass spectrometry.

14. The method of claim 12, wherein the method provides measurement of sugar monomers and sugar phosphates with femtogram detection limit in any suitable fluid or sample.

15. The method of claim 11, wherein the measuring comprises:
adding an isoamylase to a sample, the isoamylase cleaving glucose chains from glycogen;
applying a matrix-assisted laser desorption ionization (MALDI) ionization matrix to the sample; and
analyzing the samples using matrix-assisted laser desorption ionization mass spectrometry (MALDI-MS).

16. The method of claim 15, wherein the isoamylase cleaves glucose chains at only the α1,-6 linkages.

17. The method of claim 15, further comprising releasing N-linked glycans.

18. The method of claim 17, wherein releasing N-linked glycans comprises adding peptide-N-glycosidase F (PNGase F) to the sample.

19. The method of claim 15, wherein the MALDI ionization matrix is selected from the group consisting of α-cyano-4-hydroxycinnamic acid (CHCA) and DHB.

20. The method of claim 15, wherein analyzing the sample using MALDI-MS includes analyzing the sample with or without an ion-mobility enabled mass spectrometer.
